(19) United States
(12) Patent Application Publication
(10) Pub. No.: US 2015/0247792 A1
(43) Pub. Date: Sep. 3, 2015

(54) SYSTEMS AND METHODS FOR CANCER CELL DETECTION

(71) Applicant: UNIVERSITY OF NOTRE DAME, Notre Dame, IN (US)

(72) Inventors: Steven T. Ruggiero, Niles, MI (US); Carol E. Tanner, Niles, MI (US)

(21) Appl. No.: 14/636,539

(22) Filed: Mar. 3, 2015

Related U.S. Application Data
(60) Provisional application No. 61/966,748, filed on Mar. 3, 2014.

(51) Int. Cl. G01N 21/17; G06F 19/00; G01N 33/483

(52) U.S. Cl. CPC G01N 21/17; G01N 33/4833; G06F 19/345

(57) ABSTRACT

One aspect of the present disclosure relates to a system that can detect cancer. Test data can be received. The test data can include a test optical property of a preparation comprising test cells. The test data can be compared to control data. The control data can include a control optical property. Based on the comparison, a determination can be made as to whether the test cells are cancerous.

### RELATED APPLICATIONS

This application claims the benefit of U.S. Provisional Application No. 61/966,748, filed Mar. 3, 2014, entitled “Method and Apparatus for Cancerous Cell Detection,” the entirety of which is hereby incorporated by reference for all purposes.

### TECHNICAL FIELD

The present disclosure relates generally to cancer diagnostics and, more specifically, to systems and methods that can perform an optical analysis as a diagnostic tool for cancer.

### BACKGROUND

Optical techniques, which exploit significant differences in the optical properties of cancerous and healthy organs and tissues, have contributed greatly to cancer diagnostics. One example of an optical technique has seen proven value for cancer management and diagnosis on the organ and tissue level is diagnostic optical spectroscopy. In fact, diagnostic optical spectroscopy and other optical techniques have been used for cancer diagnosis and management in the brain, breast, cervix, lung, stomach, colon, prostate, and skin. However, while optical techniques have been used for cancer diagnosis and management at the organ and tissue level, such optical techniques have not been pursued at the cellular and/or sub-cellular level.

### SUMMARY

The present disclosure relates generally to cancer diagnostics and, more specifically, to systems and methods that can perform an optical analysis as a diagnostic tool for cancer. The optical analysis can detect an optical property (e.g., transmission, absorption, extinction, scattering, refraction, polarization etc.) that distinguishes normal cells and/or sub-cellular particles from cancer cells and/or sub-cellular particles. For example, the optical analysis can employ a light transmission spectroscopy (LTS) procedure to detect cancer cells or sub-cellular particles by exploiting differences in the transmission, absorption, and/or extinction of cancer cells and/or sub-cellular particles from non-cancer cells and/or sub-cellular particles.

In one aspect, the present disclosure can include a system for cancer detection. The system can include a non-transitory memory storing computer-executable instructions. The system can also include a processor that executes the computer-executable instructions. Upon the execution of the computer executable instructions, test data can be received. The test data can include a test optical property of a preparation comprising test cells. The test data can be compared to control data. The control data can include a control optical property so that the comparison can be between the test optical property and the control optical property. Based on the comparison, a determination can be made as to whether the test cells are cancerous.

In another aspect, the present disclosure can include a method for detecting cancer. The method can include steps that can be performed by a system that includes a processor. The steps can include: receiving test data that includes a test optical property of a preparation comprising test cells; comparing the test data to control data including a control optical property of a control preparation; and determining whether the test cells are cancerous based on the comparison of the test data to the control data.

In a further aspect, the present disclosure can include a method for determining whether a patient has cancer. The steps of the method can include: collecting test cells from a sample taken from the patient; collecting test cells from the sample; characterizing an optical property of the test cells using an optical characterization method; and determining whether the test cells comprise cancer based on comparing the optical property of the test cells to a control optical property. The determining step can be performed by a system that includes a processor.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 is a schematic block diagram showing a system that can detect cancer in accordance with an aspect of the present disclosure;

FIG. 2 is a schematic illustration showing transmitted light that can be input to the system shown in FIG. 1;

FIG. 3 is a schematic block diagram showing an example light transmission spectroscopy (LTS) system that can provide inputs to the system shown in FIG. 1;

FIG. 4 is a process flow diagram illustrating a method for detecting cancer in accordance with another aspect of the present disclosure;

FIG. 5 is a process flow diagram illustrating a method for determining whether a patient has cancer in accordance with another aspect of the present disclosure;

FIG. 6 shows an example overview of a sample analysis system using Light Transmission Spectroscopy (LTS) with a path length (z) of 1 cm;

FIG. 7 shows example scanning electron microscopy (SEM) images of whole normal and cancer cultured human oral cells;

FIG. 8 shows an example atomic force microscope (AFM) scan of cancer lysates in 3D mode;

FIG. 9 shows example AFM images of unfiltered cancer and normal lysates in 2D mode and associated particle histograms;

FIG. 10 shows example plots illustrating extinction data for one experimental set of lysates derived from normal cells and cancer cells;

FIG. 11 shows an example plot of optical extinction ratios as a function of inverse filter size (filter pore diameter) for cancer lysates and normal lysates;

FIG. 12 shows example plots illustrating the particle distribution density (number of particles per nm per mL) as a function of particle diameter for unfiltered cancer lysates and normal lysates; and

FIG. 13 shows example plots illustrating the number density of particles (particle count per mL) at a given particle diameter.

### DETAILED DESCRIPTION

### I. Definitions

In the context of the present disclosure, the singular forms “a,” “an” and “the” can also include the plural forms, unless the context clearly indicates otherwise. The terms “comprises” and/or “comprising,” as used herein, can specify the presence of stated features, steps, operations, elements, and/or components, but do not preclude the presence or addition of one or more other features, steps, operations, elements, components, and/or groups. As used herein, the term “and/or” can include any and all combinations of one or more of the associated listed items. Additionally, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present disclosure. The sequence of operations (or acts/steps) is not limited to the order presented in the claims or figures unless specifically indicated otherwise.

As used herein, the term “cancer” can refer to a disease caused by an uncontrolled division of abnormal cells in a part of the body. These abnormal cells can be detected and/or identified by a “diagnostic tool” that can perform an optical analysis on the cellular level.

As used herein, the term “optical analysis” can refer to the study of properties of particles suspended in a preparation related to light shining through the preparation. For example, the particles suspended in the preparation can include whole cells and/or cellular components (e.g., lysates, a mixture of substances formed by lysis of one or more cells). In some instances, the optical analysis can determine the properties of the particles based on the effects on the light as it passes through the preparation (e.g., transmission, scattering, absorption, extinction, refraction, polarization, etc.). One example of an optical analysis technique is light transmission spectroscopy (LTS), described in U.S. Pat. No. 8,456,635, which is incorporated by reference in its entirety. LTS examines the transmission, absorption, and/or extinction of light by a sample, rather than the scattering, refraction, and/or polarization of the light by the sample.

As used herein, the term “preparation” can refer to an aqueous and/or fluid suspension of cells and/or sub-cellular particles. In some instances, the preparation can include test cells (cells and/or sub-cellular particles that are taken as a sample from a patient for cancer detection). In other instances, the preparation can include a control, which can include control cells that can be used for comparison to the test cells for the cancer detection. The terms “preparation” and “medium” may be used interchangeably herein, except when referring to a computer product (e.g., a computer readable storage medium).

As used herein, the term “subject” can refer to any warm-blooded organism including, but not limited to, a human being, a pig, a rat, a mouse, a dog, a cat, a goat, a sheep, a horse, a monkey, an ape, a rabbit, a cow, etc. The terms “subject” and “patient” can be used interchangeably herein.

### II. Overview

The present disclosure relates generally to cancer diagnostics and, more specifically, to systems and methods that can perform an optical analysis as a diagnostic tool for cancer. The diagnostic tool can utilize one or more cancer cells and/or cellular components, which can exhibit an abnormal morphology and distribution of organelles, distinctly different from that of normal cells. These distinct differences can be exhibited in various optical properties. Accordingly, the optical technique can detect the differences in optical properties of the cancer cells or cellular components when compared to a control preparation (e.g., consisting of normal cells and/or cellular components or other cells with a known optical property), and these differences can be utilized as a diagnostic indicator for the presence of cancer cells. Such optical diagnosis of cancer has great value, since it can be implemented and integrated into a computer-based analytical system, and its results are straightforward and easy to interpret.

The systems and methods can be used to perform the optical analysis as the diagnostic tool for cancer using various test cells obtained from a patient. In some instances, the test cells can be cells obtained from a subject and tested for cancer. In one example, the test cells can be obtained by swabbing the inside of a patient's oral cavity at a dentist's or general practitioner's office. In other examples, the test cells can be obtained from a patient's blood plasma. In still other examples, the test cells can be obtained from a patient's biopsy from an organ or another part of the body. The test cells can be diagnosed as cancerous or non-cancerous based on comparison of optical results to known optical results of the control preparation.

### III. Systems

One aspect of the present disclosure can include a system that can detect cancer. The system can detect cancer using an optical analysis based on shining light through a preparation of one or more test cells (e.g., cells and/or or cellular components (e.g., lysates)) to investigate the intracellular morphology of the test cells to determine if the test cells are cancerous or not. For example, the system can detect optical properties present that indicate the differences in the intracellular morphology between cancer and non-cancer.

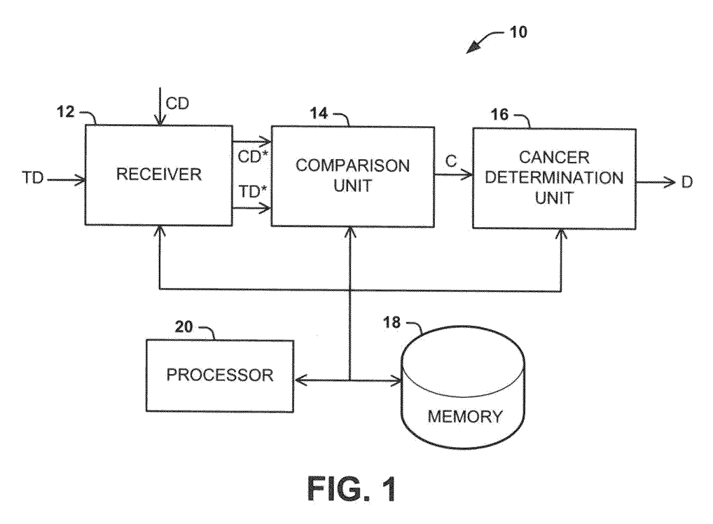

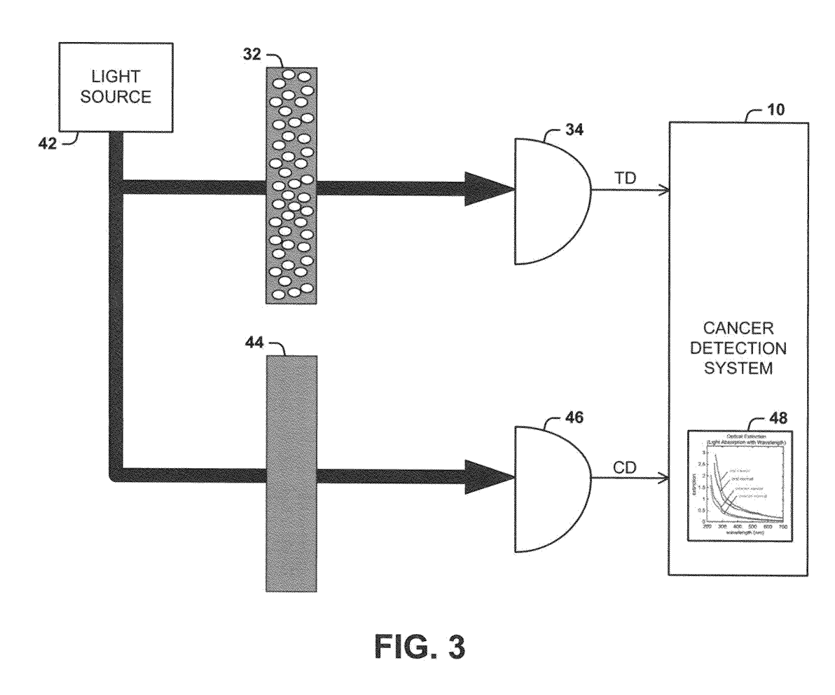

FIG. 1 illustrates an example of a system 10 that detects cancer, according to an aspect of the present disclosure. FIG. 1, as well as associated FIG. 3, is schematically illustrated as block diagrams with the different blocks representing different components. The functions of one or more of the components can be implemented by computer program instructions. These computer program instructions can be provided to a processor of a general purpose computer, special purpose computer, and/or other programmable data processing apparatus to produce a machine, such that the instructions, which execute via the processor of the computer and/or other programmable data processing apparatus, create a mechanism for implementing the functions of the components specified in the block diagrams.

These computer program instructions can also be stored in a non-transitory computer-readable memory that can direct a computer or other programmable data processing apparatus to function in a particular manner, such that the instructions stored in the non-transitory computer-readable memory produce an article of manufacture including instructions, which implement the function specified in the block diagrams and associated description.

The computer program instructions can also be loaded onto a computer or other programmable data processing apparatus to cause a series of operational steps to be performed on the computer or other programmable apparatus to produce a computer-implemented process such that the instructions that execute on the computer or other programmable apparatus provide steps for implementing the functions of the components specified in the block diagrams and the associated description.

Accordingly, the system 10 described herein can be embodied at least in part in hardware and/or in software (including firmware, resident software, micro-code, etc.). Furthermore, aspects of the system 10 can take the form of a computer program product on a computer-usable or computer-readable storage preparation having computer-usable or computer-readable program code embodied in the storage medium for use by or in connection with an instruction execution system. A computer-usable or computer-readable storage medium can be any non-transitory medium that is not a transitory signal and can contain or store the program for use by or in connection with the instruction or execution of a system, apparatus, or device. The computer-usable or computer-readable storage medium can be, for example but not limited to, an electronic, magnetic, optical, electromagnetic, infrared, or semiconductor system, apparatus or device. More specific examples (a non-exhaustive list) of the computer-readable storage medium can include the following: a portable computer diskette; a random access memory; a read-only memory; an erasable programmable read-only memory (or Flash memory); and a portable compact disc read-only memory.

As shown in FIG. 1, one aspect of the present disclosure can include a system 10 configured to detect cancer. As noted above, the system 10 can investigate intracellular morphology of one or more test cells (e.g., whole cells and/or cellular components) from an input (TD) that includes information related to an optical property (e.g., collected by an optical analysis technique) of the test cells. The system 10 can by a computer system that can receive the input (TD) and output a determination (D) of whether the test cells are cancerous. For example, the computer system can include any device that includes a non-transitory memory 18 and a processor 20, such as a laptop computer, a desktop computer, a tablet computer, a smart phone, etc.

In some instances, the system 10 can include components including at least a receiver 12, a comparison unit 14, and a cancer determination unit 16. One or more of the components can include instructions that are stored in the non-transitory memory 18 and executed by the processor 20. Each of the components can be in a communicative relationship with one or more of the other components, the processor 20, and/or the non-transitory memory 18 (e.g., via a direct or indirect electrical, electromagnetic, optical, or other type of wired or wireless communication connection), such that an action from the respective component causes an effect on the other component.

The receiver 12 can be configured to receive the input (TD). In some instances, the input (TD) can include test data including information related to the optical property of the one or more test cells of a subject. In one example, the test cells can be obtained by swabbing the inside of a patient's oral cavity at a dentist's or general practitioner's office. In other examples, the test cells can be obtained from a patient's blood plasma. In still other examples, the test cells can be obtained from a patient's biopsy from an organ or another part of the body (e.g., an ovarian biopsy, a lung biopsy, a colon biopsy, or the like).

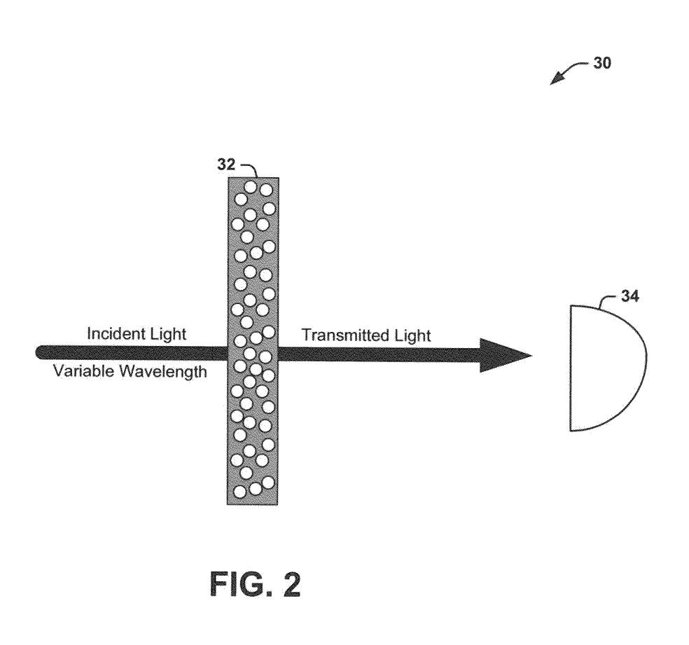

The optical property of the input (TD) can be related to an optical analysis technique. The input (TD) can include the effects on the light (e.g., transmission, scattering, absorption, refraction, polarization, etc.) as it passes through a preparation that includes the test cells (e.g., an aqueous or fluid suspension of the test cells). In some instances, the optical property can be related to the preparation of the test cells being interrogated by light at one or more discrete wavelengths. For example, the input (TD) can be related to a light transmission spectroscopy (LTS) procedure that can measure light transmitted through the preparation of the test cells (e.g., as shown in FIG. 2, light is transmitted through the preparation 32 and the transmitted light is detected by the detector 34, which sends the input (TD) to the system 10). The input (TD) recorded using LTS can have a better signal to noise ratio than other techniques. Additionally, the input (TD) recorded using LTS can measure transmission at thousands of wavelengths, so the data is more information rich than data collected using other techniques. In another example, the input (TD) can be related to a procedure that measures diffraction, scattering, or absorption of the light passing through the preparation of the test cells. The receiver 12 can perform one or more noise removal and/or signal processing procedures on the input (TD) to provide preprocessed input data (TD*) to the comparison unit 14.

Additionally, the receiver 12 can receive control data (CD) that includes a control optical property. In some instances, the control data (CD) can be related to the optical property of a control sample. For example, the control sample can be related to a preparation that includes a non-cancerous sample. In another example, the control sample can include a preparation that includes a previous (or historical) sample from the same subject that has provided the test sample. In a further example, the control sample can be related to a preparation that includes a sample from a same cell type or a different cell type from the test cell type. The control data (CD) can be recorded at the same time as the test data (TD) or at a different time than the test data. In some instances, the control data (CD) can be received from storage in the non-transitory memory 18. In other instances, the control data (CD) can be input into the receiver 12 from the same procedure as the input (TD) (e.g., a LTS procedure or another procedure that measures diffraction, scattering, or absorption of the light passing through the control sample). The receiver 12 can perform one or more noise removal and/or signal processing procedures (the same or different than the procedures performed on the test data (TD)) on the control data (CD) to provide the preprocessed control data (CD*) to the comparison unit 14.

The comparison unit 14 can receive the preprocessed test data (TD*) and the preprocessed control data (CD*). In other instances, the comparison unit 14 can receive the test data (TD) and the control data (CD) without preprocessing, by the abbreviations TD* and CD* are used for clarity of illustration.

In some instances, the test data and/or the control data can be preprocessed (e.g., by receiver 12) to have the optical property that is to be used as the diagnostic indicator emphasized to facilitate comparison by the comparison unit 14. In other instances, the comparison unit 14 can process the test data and/or the control data to emphasize the optical property that is to be used as the diagnostic indicator. For example, diagnostic indicator can be one or more specific optical properties that are distinctly different in cancer cells than in non-cancer cells. The one or more distinctly different optical properties can be due to the changes in organelle properties in the cancer cells. In some instances, the one or more optical properties can be related to transmission, extinction, absorption, scattering, refraction, polarization, etc. In other instances, the one or more optical properties can be related to transmission, extinction, and/or absorption. In still other instances, the one or more optical property can be a ratio (e.g., a ratio of extinction coefficients, transmission coefficients, absorption coefficients, scattering coefficients, or the like). For example, the ratio can take values of the coefficient at different wavelengths of light (e.g., at 250 nm and 700 nm). As another example, the ratio can take values of the coefficient for the test cells with respect to the control at the same wavelength of light.

The optical property can be related to a property of the test cells that can indicate the test cells are cancerous. In the case of the test cells including whole cells, the optical property can be related to an overall size of the cells, a mean overall size of the cells, a geometric parameter related to the cells, and/or a number of cells. In the case of the test cells including cellular components, the optical property can be related to a particle size distribution, a number of particles, and/or a particle density. The comparison unit 14 can compare the optical properties provide the comparison (C) to the cancer determination unit 16.

The cancer determination unit 16 can receive the comparison (C) and make a determination (D) as to whether the test cells exhibit cancer compared to the control sample. The determination (D) can be based on the one or more optical properties that are the diagnostic indicator. In some instances, the cancer determination unit 16 can utilize a guide or a benchmark for the optical property to make the determination (D). For example, the guide or benchmark can be developed based on historical data from patients correlating the optical property and the determination. Indeed, the data related to the optical property and the determination (D), as well as other information, can be stored in a database as additional information for the guide or benchmark. In another example, the guide or benchmark can be a known standard.

An example of a LTS system that can provide inputs (TD, CD) to the cancer detection system 10 is shown in FIG. 3. LTS can be used to measure the transmission of light through preparations of test cells 32 and control cells 44 (e.g., normal cells or other cells with a known optical property, like a previous sample from the same patient). For example, the preparations 32, 44 can include aqueous suspensions of nanoparticles in a holder made of a polymer material (e.g., polystyrene cuvettes). The preparation of test cells 32 and the preparation of control cells 44 can be in separate channels (e.g., a test channel and a reference channel). In some instances, the cells can be lysated. In other instances, the cells and/or lysates can be filtered (e.g., with a filter range of 5 microns to 0.1 microns).

The light can be produced by a light source 42 and split between the channels of the preparations 32, 44 (e.g., using a beam splitter, a mirror, or the like). In some instances, the light source 42 can provide light at a plurality of wavelengths at a high resolution. In one example example, the plurality of wavelengths can include a range from 250 nm to 1100 nm with a 1 nm resolution. However, a different wavelength range can be used at a different resolution.

LTS can measure light intensity of the transmitted beam through the preparations 32, 44 measured at zero angle with respect to the incoming beam as a function of wavelength for each of the plurality of wavelengths. Accordingly, the detectors 34, 46 can detect the transmitted beams through the preparations 32, 44 and transmit that data as inputs (TD, CD) to the cancer detection system 10. By using the control, the absolute value of the optical extinction or absorption can be obtained.

The cancer detection system 10 can use the inputs to provide a determination as to whether test cells in a preparation 32 are cancerous. The cancer detection system 10 can use the inputs (TD, CD) to determine various properties of the test cells. For example, the cancer detection system 10 can use the absolute value of the optical extinction and/or absorption to obtain the particle density (the number of particles at a given size as a function of the given particle size) using standard Mie theory and inversion with Titanov regularization. In another example, the cancer detection system 10 can use the absolute optical extinction and/or absorption to determine an absolute number of particles of a given diameter as a function of diameter (e.g., in the range from 1 nm to 3000 nm). The results from the analysis of the test cells can be compared to the results from the analysis of the control cells. In some instances, the cancer detection system 10 can use a previously-obtained guide or benchmark (e.g., element 48) or a standard guide or benchmark to facilitate the determination of whether the test sample is cancerous.

### IV. Methods

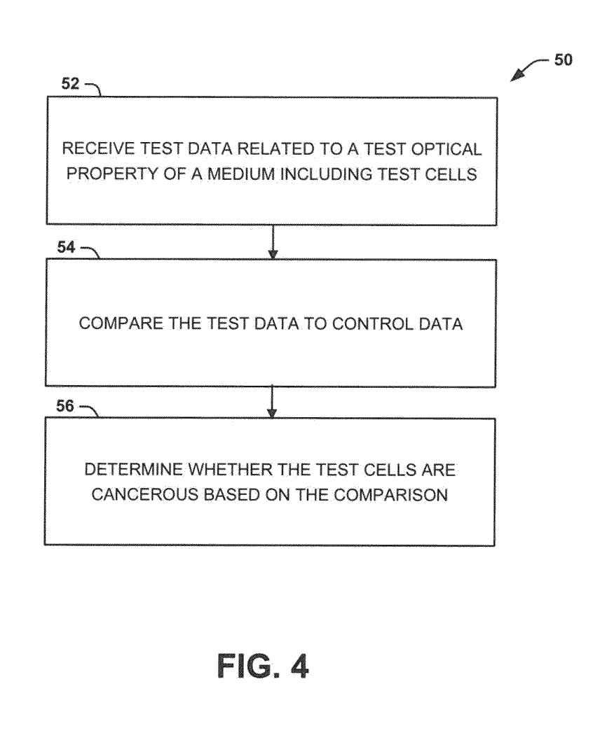

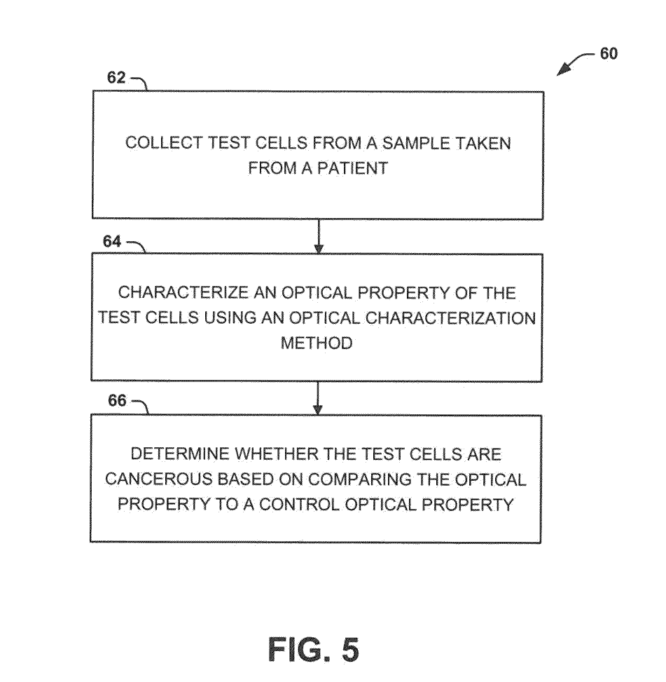

Another aspect of the present disclosure can include methods that can detect cancer. An example of a method 50 that can detect cancer is shown in FIG. 4. Another example of a method 60 that can determine whether a patient has cancer is shown in FIG. 5.

The methods 50 and 60 of FIGS. 4 and 5, respectively, are illustrated as process flow diagrams with flowchart illustrations. For purposes of simplicity, the methods 50 and 60 are shown and described as being executed serially; however, it is to be understood and appreciated that the present disclosure is not limited by the illustrated order as some steps could occur in different orders and/or concurrently with other steps shown and described herein. Moreover, not all illustrated aspects may be required to implement the methods 50 and 60.

One or more blocks of the respective flowchart illustrations, and combinations of blocks in the block flowchart illustrations, can be implemented by computer program instructions. These computer program instructions can be stored in memory and provided to a processor of a general purpose computer, special purpose computer, and/or other programmable data processing apparatus to produce a machine, such that the instructions, which execute via the processor of the computer and/or other programmable data processing apparatus, create mechanisms for implementing the steps/acts specified in the flowchart blocks and/or the associated description. In other words, the steps/acts can be implemented by a system comprising a processor that can access the computer-executable instructions that are stored in a non-transitory memory.

The methods 50 and 60 of the present disclosure may be embodied in hardware and/or in software (including firmware, resident software, micro-code, etc.). Furthermore, aspects of the present disclosure may take the form of a computer program product on a computer-usable or computer-readable storage medium having computer-usable or computer-readable program code embodied in the storage medium for use by or in connection with an instruction execution system. A computer-usable or computer-readable storage medium may be any non-transitory storage medium that can contain or store the program for use by or in connection with the instruction or execution of a system, apparatus, or device.

Referring to FIG. 4, an aspect of the present disclosure can include a method 50 for detecting cancer. The method 50 can exploit distinct differences in one or more optical properties of cancer versus normal cells to create a diagnostic tool for cancer. For example, the optical properties can be related to transmission, absorption, scattering, extinction, and/or diffraction of light by the cells. As another example, the optical properties can be related to transmission, absorption, and/or extinction of light by the cells. As a further example, the optical properties can be related to a ratio of coefficients related to transmission, absorption, scattering, extinction, and/or diffraction of light by the cells.

At 52, test data (e.g., TD) related to a test optical property of a preparation (e.g., an aqueous or fluid suspension) including test cells can be received (e.g., by receiver 12). In some instances, the test data can include information related to the optical property of the test cells (e.g., whole cells and/or cellular components). For example, the test data can be taken using light transmission spectroscopy. In another example, the test data can be taken using another method including diffraction, absorption, and/or scattering of light.

At 54, the test data can be compared (e.g., by comparison unit 14) to control data (e.g., CD). For example, the control data can include a control optical property related to a control sample. For example, the control sample can be related to a preparation that includes a non-cancerous sample. In another example, the control sample can include a preparation that includes a previous (or historical) sample from the same subject that has provided the test sample. In a further example, the control sample can be related to a preparation that includes a sample from a same cell type or a different cell type from the test cell type. In some instances, the control data can be recorded at the same time as the test data or at a different time than the test data. In other instances, the control data can stored in a memory device.

At 56, it can be determined whether the test cells are cancerous based on the comparison (e.g., by cancer determination unit). The determination can be based on the optical property that is the diagnostic indicator. In some instances, the determination can be based on a guide or a benchmark for the optical property. For example, the guide or benchmark can be developed based on historical data from patients correlating the optical property and the determination. In another example, the guide or benchmark can be a known standard.

Referring now to FIG. 5, another aspect of the present disclosure can include a method 60 for determining whether a patient has cancer. At 62, test cells can be collected from a sample taken from a patient. In some instances, the cells can be cultured and harvested at a later date after they are collected. In other instances, the cells can be pretreated and isolated but otherwise left with their cell membrane intact. In other instances, the cells can undergo lysis and/or homogenation so the lysates can be analyzed. The cell sample can undergo one or more filtration steps (e.g., at a filter size in the range of 5 microns to 0.1 microns).

At 64, an optical property of the test cells can be characterized using an optical characterization method (e.g., LTS or another optical characterization method). For example, the optical property can be related to transmission, absorption, scattering, extinction, and/or diffraction of light by the cells. As another example, the optical property can be related to transmission, absorption, and/or extinction of light by the cells. As a further example, the optical property can be related to a ratio of coefficients related to transmission, absorption, scattering, extinction, and/or diffraction of light by the cells.

At 66, it can be determined whether the test cells are cancerous based on comparing the optical property to a control optical property. The determination can be based on the optical property that is the diagnostic indicator. In some instances, the determination can be based on a guide or a benchmark for the optical property. For example, the guide or benchmark can be developed based on historical data from patients correlating the optical property and the determination. In another example, the guide or benchmark can be a known standard.

### V. Examples

The following examples are for the purpose of illustration only and are not intended to limit the scope of the appended claims.

### Example 1

This example illustrates the optical properties of lysates from cultured human oral cancer and normal cells. To demonstrate the optical properties, experiments were completed on the cultured human oral cancer and normal cells using light transmission spectroscopy (LTS).

### Methods

### Lysate Specimens

Cultured cancer and normal human oral cells were collected from culture plates and then transferred to microtubes with a 0.1 M phosphate-buffered saline (PBS) solution. Samples were divided in half: one half was used for whole-cell measurements, and the other half was lysed. Cell lysates were prepared at room temperature using a standard Dounce homogenizer. After being homogenized with 30 strokes of the pestle, cell lysate material was transferred into microtubes without filtration. For this work normal and cancer lysates were further diluted by factors of 12 and 24, respectively, to create similar overall light transmission properties.

### Scanning Electron Microscopy

For scanning electron microscope (SEM) characterization, cells underwent treatment through fixation, dehydration, drying and coating. First, isolated cells were added to the first fixative, a 2% glutaraldehyde buffer solution, for one hour. After three PBS rinses, cell samples were added into the second fixative, a 1% osmium tetroxide buffered solution, for another hour followed by three PBS rinses. Then, fixed cell samples were dehydrated using a graded series of ethanol solutions in DI water: 50%, 70%, 80%, 95%, and 100%, and finally collected on filter paper for critical-point drying using 100% liquid CO₂. To enhance surface electrical conductivity, the samples were coated with a 20 nm film of sputtered iridium. Imaging was performed using a Magellan 400 field-emission scanning-electron microscope.

### Atomic-Force Microscopy

For atomic force microscopy, a 1×1 inch piece of mica was cleaved and transferred to a nitrogen-purged case. Prior to use, the mica was washed with methanol for at least 15 seconds and then rinsed with di-ionized (DI) water. A drop of the lysate sample was then pipetted on to the mica to allow particles to adsorb to the surface. The sample was then rewashed with DI water and dried with dry nitrogen. For AFM imaging, a Park Systems XE-70 AFM model was used in non-contact in-air mode, using a non-contact tip. The stated resolution of an unused non-contact tip is 2-3 nm.

### Light Transmission Spectroscopy (LTS)

The basic operational principle of light transmission spectroscopy (LTS) is that the intensity of a transmitted light beam as a function of wavelength is measured at zero angle with respect to the incoming beam after it passes through a liquid suspension of nanoparticles. By also using a reference channel containing the suspension fluid, the absolute value of the optical extinction due to the particles can be obtained over a broad spectral range (250 to 1100 nm) at high (1 nm) resolution. LTS also uses the measured wavelength-dependent optical extinction to obtain the particle distribution density (number of particles of a given diameter per unit diameter per unit volume, as a function of particle diameter) by performing spectral inversion using a Mie solution to Maxwell's equations, which can provide the number density (total number of objects of a given diameter per unit volume) as a function of particle diameter, over a range of 1-3000 nm.

### LTS Apparatus

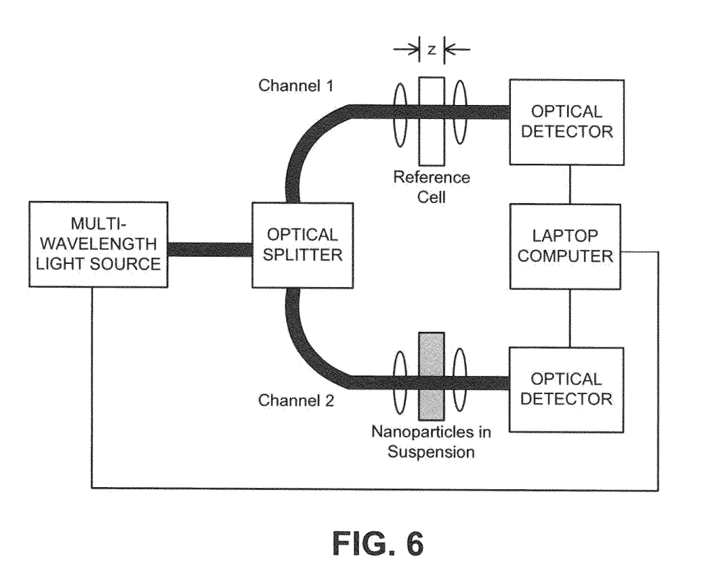

The LTS apparatus is shown schematically in FIG. 6. Measurements took place in the following manner to remove any differences in the optical path or fluctuations in the light source while allowing for the elimination of the effect of the optical extinction of the suspension material itself, leaving the extinction of the suspended lysate nanoparticles exclusively. After mechanical processing, the lysates were diluted to produce significant light extinctions from ˜250 to 1000 nm. A given lysate sample was pipetted into an optical cuvette and then placed in one optical channel of the LTS spectrometer and the suspension material (PBS) was pipetted into an identical optical cuvette and placed in the second channel. After a spectrum of both samples was obtained (taking a few seconds), the cuvettes were exchanged and a second spectrum was obtained of both samples. The optical extinction was measured,

### Results

### Cell Characterization by SEM

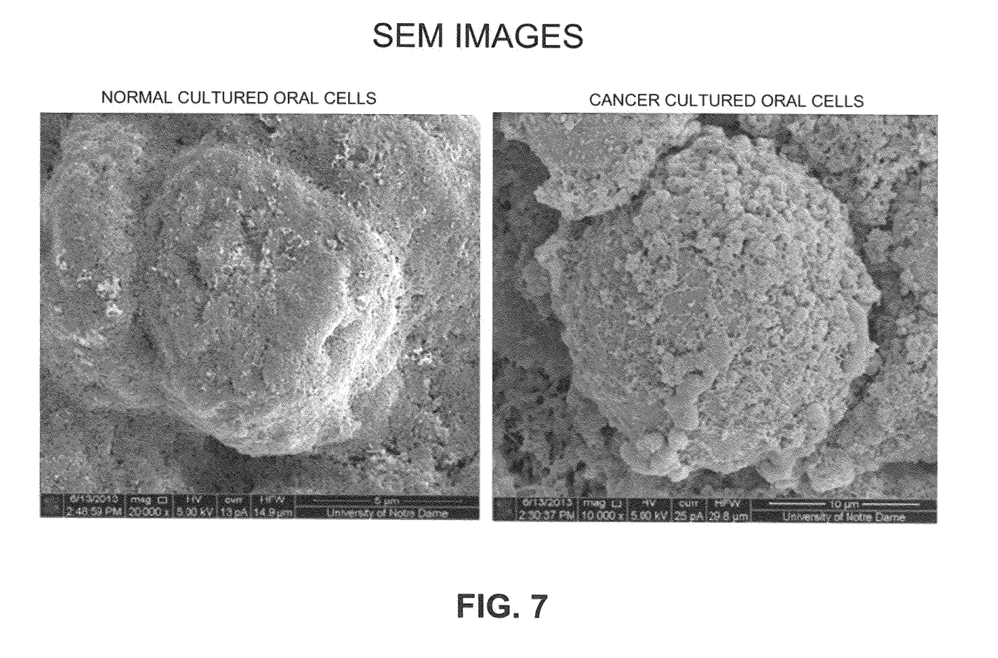

Shown in FIG. 7 are representative SEM images typical of individual whole human oral normal and cancer cells used in this study. The cancer cells we examined were generally larger in diameter (˜20 μm) compared to normal cells (˜10 μm) and had a less smooth surface.

### Lysate Characterization by AFM

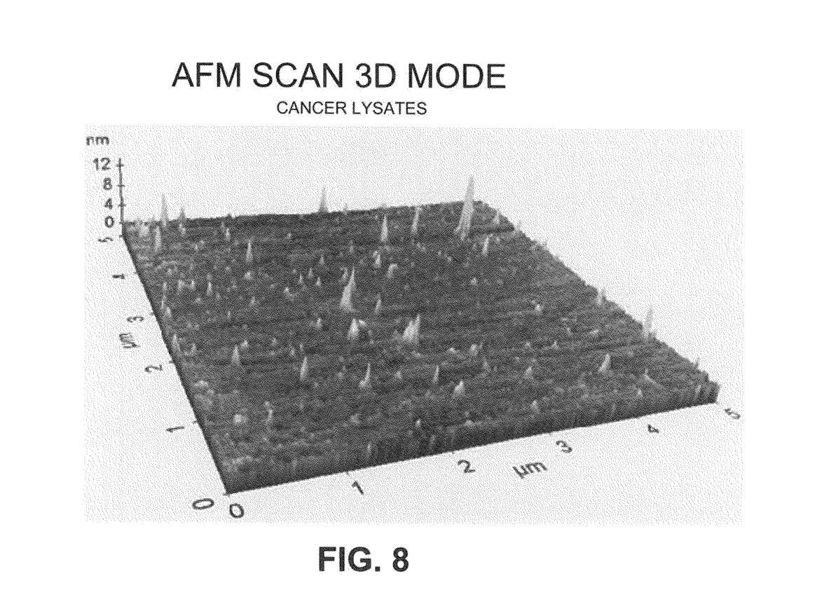

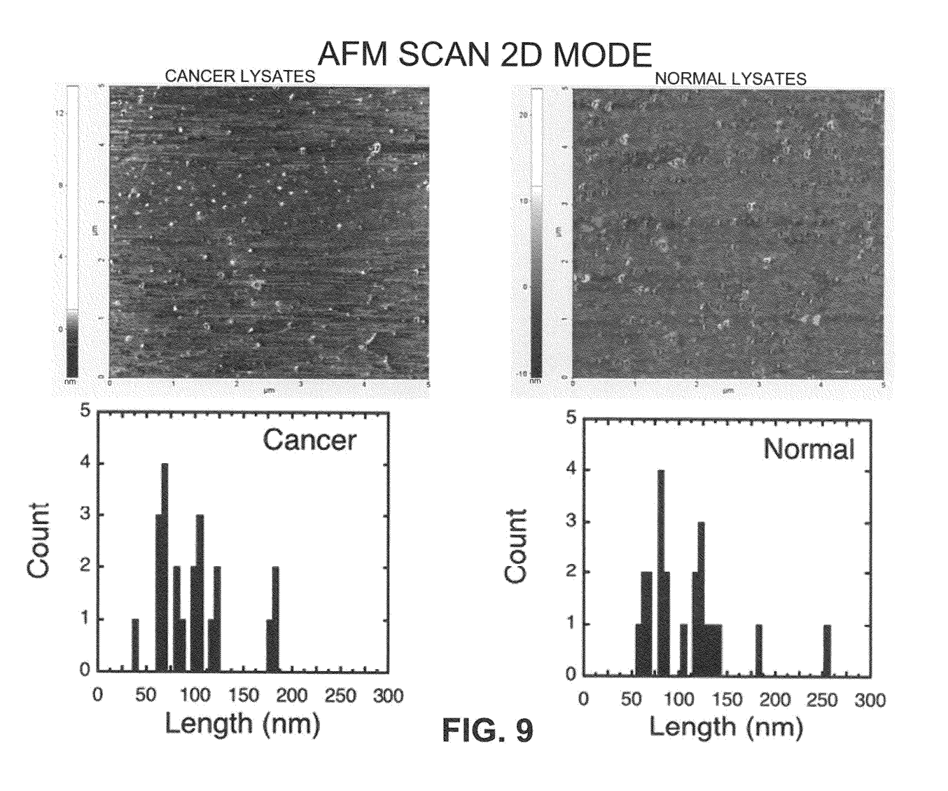

An AFM scan of unfiltered cancer lysates rendered in three dimensions is shown in FIG. 8. FIG. 9 shows (1) more quantitative AFM scans of cancer and normal lysates in 2D mode and (2) the image-analysis based distributions of the measured particle lengths. The mean particle sizes for cancer (99±40 nm) and normal (108±46 nm) lysates are essentially identical.

### Lysate Characterization by LTS

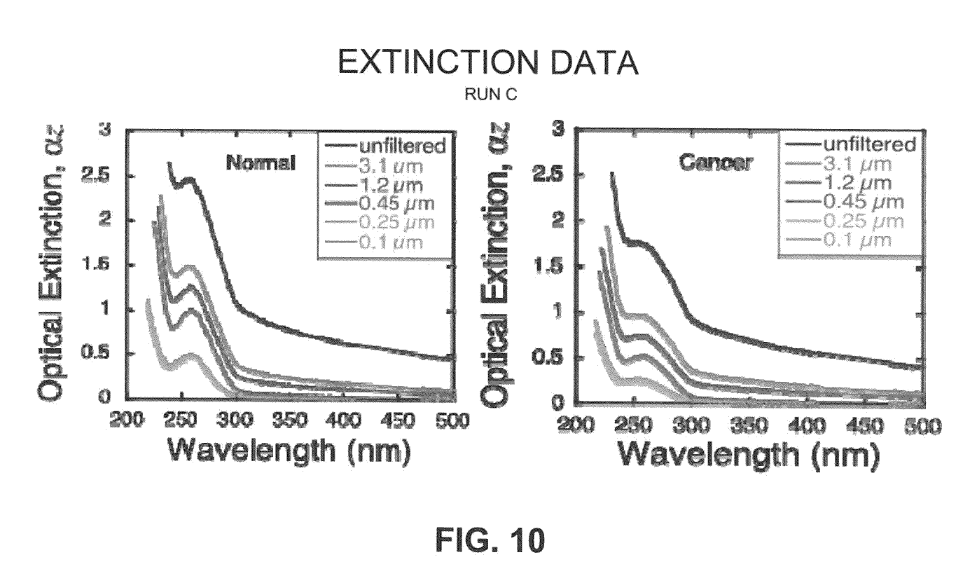

Unfiltered lysate material in PBS was initially measured and then this material was filtered by a succession of standard syringe filters and measured by LTS at each filtration stage. For these experiments, the absolute densities and growth rates of cancer and normal cells dictated that the normal-cell-derived lysates and cancer-cell-derived lysates were diluted by factors of 12 and 24, respectively, to achieve comparable optical extinction properties. Three distinct sets of cancer and normal cells were grown on separate occasions, referred to as runs A, B, and C. The optical extinction is shown in FIG. 10 for one pair of normal and cancer cells (run C) as a function of filtration.

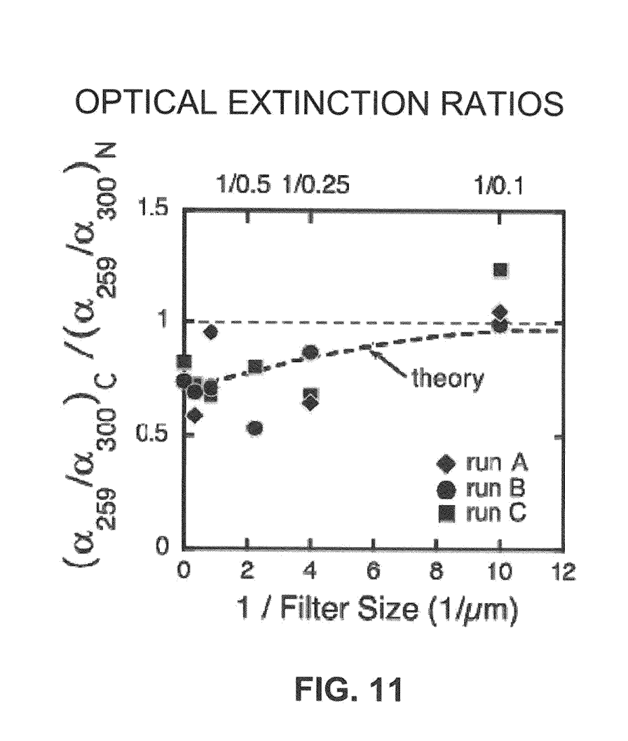

The comparative degree of the optical extinction between cancer and normal lysates was determined qualitatively by appropriately normalizing the data for any overall differences in the particle densities of the lysates. For each filtration (and for unfiltered samples), value of the extinction at 259 nm was normalized to the value of the extinction at 300 nm, to give the ratio (α₂₅₉/α₃₀₀)_{C} for cancer cells and (α₂₅₉/α₃₀₀)_{N} for normal cells. This quantity is designed to characterize the relative degree of UV extinction, represented by the peak value of the extinction seen consistently at 259 nm. Although the peak is likely associated with the presence of DNA, it is a convenient fiducial point and generally scales with the more dominant background UV absorption. This is normalized by the “knee” value at 300 nm that characterizes the extinction behavior at longer wavelengths. Using this procedure, the overall extinction results with systematic filtration are shown in FIG. 11. Based on the results shown in FIG. 11, the normal lysates have particle distributions that have higher quantities of smaller particles than larger particles, as compared with cancer lysates, thus generally making (α₂₅₉/α₃₀₀)_{C}/(α₂₅₉/α₃₀₀)_{N})<1 (true for all filtrations except for 0.1 μm).

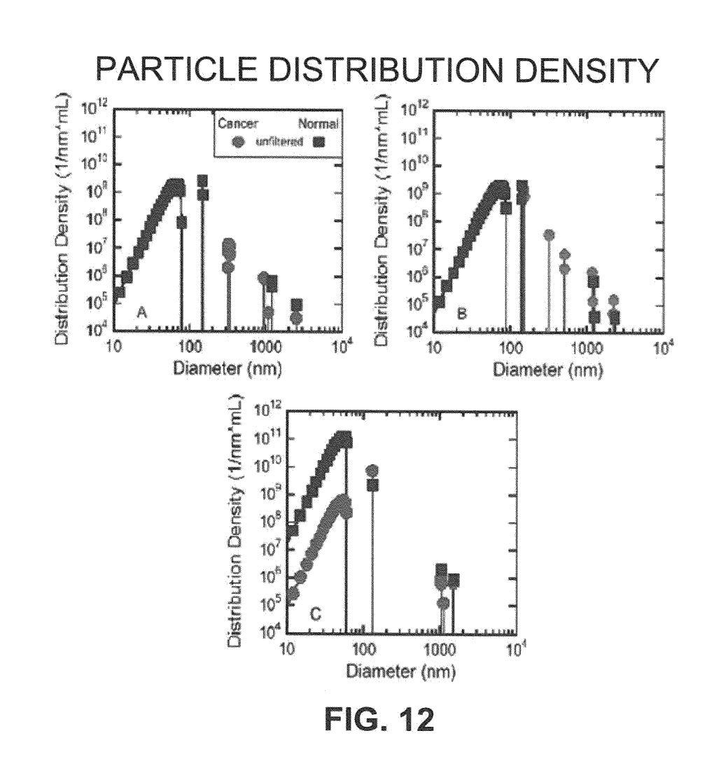

Shown in FIG. 12 is the particle distribution density (the number of particles per mL per nm) for three runs of cancer/normal lysate samples as determined by LTS plotted on a log-log scale because of the large dynamic range available with LTS. The plot of FIG. 12 was created with the afore-mentioned Mie inversion procedure using data of the type presented in FIG. 11. These results are consistent with the AFM results in showing a peak in the particle density in the vicinity of 100 nm. What is apparent from these results is: (1) that there are many more particles at small particle sizes, especially below ˜300 nm; (2) that generally normal lysates have a larger number of smaller particles; and (3) that the number of larger particles is roughly equal for cancer and normal particles.

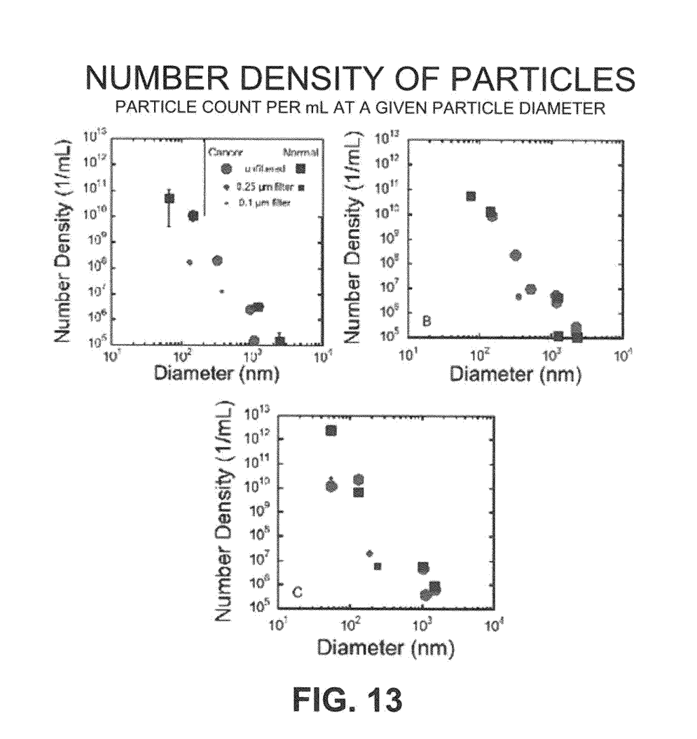

The distribution number density of the particles is shown in FIG. 12 versus the diameter (maximum value for a given peak in the distribution density), found by taking the area under the peaks of FIG. 12. In FIG. 13, there is a clear dominance of smaller particles in the normal lysates for unfiltered samples (large symbols), and a general demarcation point of ˜300 nm for the boundary between the far higher populations of smaller particles and those with larger size as noted above in connection with FIG. 12. As filtering proceeds to the smallest size scales (small symbols), this imbalance becomes less evident. Also evident is the power-law dependence of the results shown in FIG. 13.

From the above description, those skilled in the art will perceive improvements, changes and modifications. Such improvements, changes and modifications are within the skill of one in the art and are intended to be covered by the appended claims.

What is claimed is:

1. A system for cancer detection, the system comprising:
a non-transitory memory storing computer-executable instructions; and
a processor that executes the computer-executable instructions to at least:
receive test data comprising a test optical property of a preparation comprising test cells; and
compare the test data to control data comprising a control optical property; and
determine whether the test cells are cancerous based on the comparison.

2. The system of claim 1, wherein the test optical property is related to at least one of transmission, absorption, scattering, and extinction of light by the preparation.

3. The system of claim 1, wherein the comparison of the test data to the control data is based on a ratio of extinction coefficients.

4. The system of claim 1, wherein the test cells comprise whole cells or cellular components.

5. The system of claim 1, wherein the test data corresponds to interrogation by light comprising one or more discrete wavelengths.

6. The system of claim 1, wherein the control optical property is obtained from a preparation comprising a non-cancerous sample.

7. The system of claim 1, wherein the control optical property is obtained from a preparation comprising a previous sample from a patient that provided the test sample.

8. The system of claim 1, wherein the control optical property is received from a preparation comprising a sample from at least one of a same cell type as the test sample and a different cell type from the cell type of the test sample.

9. The system of claim 1, wherein the preparation comprising the test cells undergoes a light transmission spectroscopy procedure to produce the test data.

10. A method for cancer detection, the method comprising the steps of:
receiving, by a system comprising a processor, test data comprising a test optical property of a preparation comprising test cells; and
comparing, by the system, the test data to control data comprising a control optical property of a control preparation; and
determining whether the test cells are cancerous based on the comparison.

11. The method of claim 10, wherein the test optical property comprises an extinction coefficient.

12. The method of claim 10, wherein the test optical property is related to at least one of a particle size distribution, a number of particles, and a particle density.

13. The method of claim 10, wherein the test optical property is related to at least one of an overall size and a geometric parameter.

14. The method of claim 10, wherein the test optical property comprises at least one of an absorption coefficient and a scattering coefficient.

15. The method of claim 10, the preparation comprising the test cells undergoes an optical data acquisition procedure to acquire the test data.

16. The method of claim 15, wherein the optical data acquisition procedure comprises light transmission spectroscopy.

17. A method for determining whether a patient has cancer, the method comprising:
collecting test cells from a sample taken from the patient;
characterizing an optical property of the test cells using an optical characterization method; and
determining, by a system comprising a processor, whether the test cells comprise cancer based on comparing the optical property of the test cells to a control optical property.

18. The method of claim 17, further comprising lysating the test cells before the characterization.

19. The method of claim 17, further comprising filtering the test cells a plurality of times,
wherein the characterizing is performed for each of the plurality of filtrations.

20. The method of claim 17, wherein the optical characterization method comprises light transmission spectroscopy.
